(19) United States
(12) Patent Application Publication
(10) Pub. No.: US 2007/0071686 A1
(43) Pub. Date: Mar. 29, 2007

(54) Liquid preparation containing tobramycin

(75) Inventors: Frank-Christophe Lintz, Stolberg (DE); Manfred Keller, Munich (DE)

Correspondence Address: EDWARDS & ANGELL, LLP, P.O. BOX 55874, BOSTON, MA, 02205, US

(73) Assignee: Pari GmbH, Starnberg (DE)

(21) Appl. No.: 10/575,668

(22) PCT Filed: Oct. 14, 2004
(86) PCT No.: PCT/EP04/11572

§ 371 Date: Apr. 14, 2006

(30) Foreign Application Priority Data
Oct. 15, 2003 (DE) 103 47 995.3

(51) Int. Cl. A61K 31/7034; A61K 9/12

(52) U.S. Cl. 424/45; 514/35; 514/38

(57) ABSTRACT

The application describes a sterile aqueous inhalation solution containing the active agent tobramycin. The preparation has a high content of active agent (about 80 to 120 mg/ml of tobramycin) and contains an acidic adjuvant, but contains only a low concentration of sodium chloride (at most about 2 mg/ml). It can be injected or administered as an aerosol, for example with conventional nebuliser. It is particularly suitable for application in combination with a modern vibrating membrane nebuliser and allows the administration of a therapeutic single does in markedly less than 10 minutes.

### TECHNICAL FIELD OF THE INVENTION

The invention relates to liquid preparations which contain the antibiotic tobramycin and which can be administered as pharmaceutical preparations by injection or as an aerosol, namely pulmonarily or nasally. Furthermore, it relates to pharmaceutical kits comprising two components from which liquid preparations for administration of tobramycin can be prepared. Moreover, the invention relates to the use of the preparations in pharmaceutical products which can be administered pulmonarily or nasally by means of a nebuliser and which can be employed for the treatment of cystic fibrosis or other infectious diseases of the respiratory tract.

### BACKGROUND OF THE INVENTION

Tobramycin is an aminoglycoside antibiotic which is chemically designated as O-3-amino-3-desoxy-α-D-glucopyranosyl(1→4)-O-[2,6-diamino-2,3,6-tridesoxy-α-D-ribo-hexopyranosyl-(1→6)]-2-deoxystreptamine, which is employed systemically and locally for the treatment of serious infections. Systemic treatment is carried out by injection or infusion; this is indicated in the case of serious infections with a number of tobramycin-sensitive Gram-negative bacteria, in particular, in the case of septicaemia, infections of the lower respiratory tract, of the urogenital systems, intraabdominal infections, infections of the skin, soft tissues and bones, osteomyelitis, purulent arthritis, bacterial endocarditis, Gram-negative meningitis as well as in infections in immunosuppressed patients.

In the case of serious infections of the respiratory tract, tobramycin can be administered by inhalation. Thus, for example, in Germany, the medicament TOBI (marketed by Chiron) is available which contains tobramycin in the form of an aqueous solution which is free of antioxidants and which can be inhaled as an aerosol. In this form, the active agent can be used for the treatment of infections of the lower respiratory tract with Pseudomonas aeruginosa in patients with mucoviscidosis or cystic fibrosis. Further areas of application for which clinical tests are being carried out include the therapy of bacterial infections in cases of bronchiectasia and in respirated patients as well as in cases of tuberculosis.

Mucoviscidosis (or cystic fibrosis) is one of the most common congenital metabolic disorders. It is an autosomally recessively inherited multi organ syndrome caused by a lack of CFTR (cystic fibrosis transmembrane regulator), a regulatory protein of chloride transport through cell membranes with a resulting increase in a viscosity of bodily secretions. The enzyme defect is located on chromosome 7. The gene defect affects, in particular, exocrine glands, as a consequence of which many organs form a viscous mucus which, as it were, blocks the lungs, the pancreas and the biliary tract. About 90 percent of the problems relates to the respiratory organ. A chronic pneumonia frequently results when the viscous mucus impedes the removal of bacteria. Especially Pseudomonas aeruginosa tend to affect the lungs of patients with mucoviscidosis. This results in a kind of vicious circle: The growth and reproduction of the bacteria increases the secretion of mucus and results in infection and inflammation of the respiratory tract and it becomes all the more difficult to provide oxygen to the air passages. In many mucoviscidosis patients, the chronic inflammation of the lungs results in a progressive destruction of the lung tissues and to serious breathing disorders from which, eventually, over 90% of the patients die.

### STATE OF THE ART

The pulmonary treatment of mucoviscidosis patients with the active agent tobramycin is currently mainly carried out with the medicament TOBI. In contrast to injectable preparations of tobramycin, TOBI contains no stabilising addition of antioxidants, which, upon inhalation, can trigger fits of coughing or asthma.

TOBI is aerosolised and inhaled by means of a nebuliser. Depending on the construction and type of nebuliser, strongly varying results are achieved. The efficiency of the pulmonary administration depends particularly strongly on the size of the particles of the aerosol that is produced which varies greatly with the device used. The pharmaceutical manufacturer Chiron especially recommends, for therapy with TOBI, the jet nebuliser PARI LC PLUS™ in combination with the compressor Pari Master™ (both marketed by PARI).

The literature describes further preparations of tobramycin for inhalation. U.S. Pat. No. 5,508,269 describes a preparation with about 200 mg to 400 mg of tobramycin in a volume of about 5 ml. As an isotonising adjuvant, this contains sodium chloride in an amount of about 0.225% and the pH of the preparation is adjusted to about 5.5 to 6.5. For administration, the preparation is to be nebulised by means of a jet or ultrasonic nebuliser to produce an aerosol with a particle size of 1 to 5 μm.

U.S. Pat. No. 6,083,922 describes rather similar preparations of the active agent tobramycin which are, however, to be employed for the treatment of infections with Mycobacterium tuberculosis. About 80 to 300 mg of the active agent are to be added as a single dose to a volume of about 3 to 5 ml. The pH is to be adjusted to 5.5 to 7.0 and sodium chloride is again used for isotonisation.

The preparation of tobramycin described in U.S. Pat. No. 6,387,886 also has a very similar composition. It contains about 250 to 350 mg of the active agent in 5 ml of sodium chloride solution, the pH of which is again adjusted to 5.5 to 6.5. The proposed use is a therapy of chronic bronchitis with tobramycin-sensitive pathogens.

WO 03/004005 describes a preparation with a tobramycin content of 75 mg/ml and a sodium chloride concentration of 0.45%. Unlike the documents cited above, this publication requires a pH between 4.0 and 5.5. As a further feature, an osmotic pressure in the range of 250 to 450 mOsmol/l is indicated. In the preferred embodiment, the preparation has a pH of 5.2 and an osmotic pressure of 280 to 350 mOsmol/l.

In practice, all these tobramycin preparations exhibit various disadvantages. Firstly, their compatibility is not particularly satisfactory. This is probably caused by the active agent itself and exacerbated by the affected state of the respiratory tract of the mucoviscidosis patients. Secondly, it takes patients quite a long time to inhale a single of the active agent (the inhalation of 300 mg of tobramycin in 5 ml of liquid is currently most common), namely about 15-20 minutes when using common jet nebuliser (depending on the device). Especially for serious ill patients this can represent a serious burden. A further disadvantage of conventional preparations is their taste, which many patients perceive as bad; of course, this is predominantly caused by the active agent, i.e., by those aerosol droplets which impact in the mouth and larynges and subsequently—mixed with saliva—reach the taste buds of the tongue. This is what happens to a considerably proportion of the inhaled aerosol droplets.

In order to address at least the problem of long inhalation times and the burden on patients resulting therefrom, WO 02/094217 suggests to use a more concentrated tobramycin solution, with which a single dose can be inhaled more quickly due to the lower volume. The application volume is to be reduced to not more than 4, preferably to not more than 3.5 ml. The concentration of the active agent, in turn, is to be increased to about 200 mg/ml so that an inhalation time of less than 10 minutes is achieved. A concentration of the active agent of 90 to 120 mg/ml and an inhalation time of less than about 6 minutes is particularly preferred. The latter, however, is also to be achieved by employing, instead of conventional nebulisers, modern devices with a particularly high aerosol output. There are recommended, for example, stronger compressors which can be connected to conventional jet nebulisers, or piezoelectric nebulisers which, due to their functional principle, show greater performance. However, in the rather detailed discussion of the examples, the document describes only a single preparation which has a concentration of the active agent of more than 60 mg/ml, namely a preparation with 420 mg of tobramycin in 3.5 ml, which corresponds to a concentration of the active agent of 120 mg/ml. At the same time, this preparation contains an unspecified adjuvant for adjusting the pH to 6.0±0.5 as well as 0.225% of sodium chloride. However, it turned out that this preparation could not be applied efficiently with the selected means which were optimised for a short inhalation time. In a clinical study, the amount of active agent retrieved in plasma and saliva was not greater than after inhalation of 300 mg of tobramycin in the form of the medicament TOBI. Thus, the inhalation time could be reduced, compared to TOBI (300 mg), from 18.1 to 9.7 minutes, but only at the expense of bioavailability.

One disadvantage of known preparations of tobramycin for inhalation is the non-optimal compatibility in the respiratory tract. Compared to the application of a nebulised placebo solution, reactions such as coughing and irradiation of the respiratory tract are observed more frequently upon inhalation of TOBI or the known experimental tobramycin preparations. It has not yet been entirely resolved whether this is purely an effect of the active agent, which can hardly be influenced, or whether the combination with certain common adjuvants contributes to the incompatibility or might contribute to its reduction.

### DESCRIPTION OF THE INVENTION

Thus, there is a need for preparations of tobramycin for the efficient, patient-compatible, effective and compatible inhalation. In particular, there is a need for preparations of this active agent which can be administered quickly and efficiently with high performance inhalers and are well tolerated and which do not have the disadvantages of known preparations. It is the object of the invention to provide such improved preparations.

This object is achieved by the provision of preparations according to claim 1. Further solutions will become apparent from the other claims and from the following description. The preparations can improve the pulmonary antibiotic therapy of mucoviscidosis patients; however, they can also be employed as solutions for injection or for the local treatment of infections affecting the upper respiratory tract.

There is claimed a sterile, liquid preparation in the form of an aqueous solution for injection or inhalation, which contains about 80 to 120 mg of tobramycin per ml and, additionally, an acidic adjuvant and a concentration of sodium chloride of at most about 2 mg/ml.

In this context, an aqueous solution means a solution or colloidal solution, the solvent of which consists entirely or predominantly of water. Sterile means that the preparation complies, with respect to its sterility, to the requirements of the European pharmacopoeia (Pharm. Eur.) as may be applicable. Tobramycin is the substance O-3-amino-3-desoxy-α-D-glucopyranosyl(1→4)-O-[2,6-diamino-2,3,6-tridesoxy-α-D-ribo-hexopyranosyl-(1 →6)]-2-deoxystreptamine including its salts, complexes, conjugates and derivatives. The stated concentration of about 80 to 120 mg/ml, however, refers to the base of tobramycin. It is to be noted that, in practice, slight deviations from the nominal concentration do of course occur, which are absolutely common and tolerable. Thus, for example, for a preparation with a nominal concentration of 80 mg/ml, an actual concentration of 78.5 mg/ml may well be within the product specification. Accordingly, a pharmaceutically tolerable deviation at a concentration of active agent in the range of 80 to 120 mg/ml is comprised by the invention.

The acidic adjuvant is a physiologically acceptable acid or an acidic salt with which the pH of the preparation is adjusted. According to the invention, sodium chloride is either not present at all or only at a concentration of at most 2 mg/ml, wherein the same tolerances as with respect to the active agent apply.

It has been found that the preparations formulated according to claim 1 are excellently useful to be aerosolised in common nebulisers. The aerosols can be inhaled rapidly and efficiently. In particular, in combination with the optional features which are described below, a markedly improved patient-compatible therapy of pulmonary infections in cases of mucoviscidosis can be achieved.

In order to achieve the aim of a convenient, safe and efficient inhalation of a therapeutic dose of tobramycin, various parameters need to be taken into account, some of which are of a formulation-related kind. One decisive parameter is the concentration of the active agent in the inhalation solution. The marketed product TOBI, at 300 mg/5 ml, has a markedly lower concentration of the active agent than is required according to the present invention. Due to the low concentration, it is hardly possible to administer the inhalation solution TOBI within a short inhalation time. While an inhalation time of at most about 6-8 minutes would appear desirable, and an inhalation time of at most 4-5 minutes is particularly desirable, in order to achieve high patient-compliance, the inhalation of the 5 ml of TOBI solution in combination with the nebuliser recommended in the instructions for use requires at least about 15-20 minutes. Even if this recommendation is ignored and a more powerful nebuliser is used, the desirable inhalation time can hardly be achieved since, because of the low concentration of active agent, a relatively large amount of liquid must be nebulised.

Surprisingly, it was now found that the concentration of active agent should not be selected arbitrarily high within the limits of solubility of the active agent, but that a value of about 120 mg/ml should not be exceeded. Thus, it was found that, at an increasing concentration of tobramycin, the surface tension of the solution may well be kept within the desired range of about 70-76 mN/m, but that the dynamic viscosity, which is equally relevant for nebulisation, increases markedly and has a negative effect on nebulisation. Thus, aqueous solutions of tobramycin with a pH of 6.0 to 6.5 have a 50% greater viscosity (about 2.9 mPa·s) at a concentration of active agent of 180 mg/ml compared to a solution with 100 mg/ml (about 1.8 mPa·s). ComParable solutions with 120 mg/ml of tobramycin have a viscosity of about 2.1 mPa·s and can still be nebulised nearly as efficiently as solutions with 100 mg/ml. Under standard conditions, the preparations according to the present invention have a viscosity of about 1.4 to 2.3 mPa·s, and preferably a viscosity in the range of about 1.6 to 2.0 mPa·s. A viscosity of about 1.8 mPa·s is most preferred.

The selection and amount of the isotonising agent has a special influence on the local compatibility of the preparation. The sodium chloride contained in the marketed product TOBI is also used or recommended in nearly all of the preparations described in the literature, for example, in WO 03/004005, where 0.45% (w/v) of sodium chloride are used, and in WO 02/094217, where preferably 0.225% (w/v) are employed. However, inhalation experiments carried out by the inventors showed that a low sodium chloride concentration between 0.0 and 0.2% (w/v) is less irritating and optimally compatible with the other mandatory and optional ingredients. In one of the preferred embodiments of the invention, sodium chloride is present at a concentration of less than 0.2% (w/v), preferably at a concentration of 0.17% (w/v). In a further embodiment, no sodium chloride is present, except for ubiquitous amounts of sodium chloride which may also be contained in water of pharmaceutical quality. In another embodiment, an essentially neutral salt is contained in the preparation as isotonising agent which salt is not sodium chloride but, for example, a sodium sulphate or sodium phosphate. In this case, however, salts other than sodium salts are even more preferred. Thus, it is known of certain calcium and magnesium salts that they can have a positive or supportive effect in the inhalation of solutions of active agents, possibly because they themselves counteract the local irritation caused by the administration and have a bronchodilatory effect which is currently postulated in the clinical literature (see, for example, R. Hughes et al., Lancet. 2003; 361 (9375): 2114-7) and/or because they inhibit the adhesion of germs to the proteoglycans of the mucosa of the respiratory tract such that the mucociliary clearance is indirectly supported as a natural defense mechanism of the organism against the germs (K. W. Tsang et al., Eur. Resp. 2003, 21, 932-938). Magnesium sulphate, which as excellent pulmonary compatibility and can be inhaled without fear of problems, as well as calcium chloride (1-10 mMol) are particularly preferred. If the latter effect is to be reinforced, the use of heparin or phytohemaglutinine can be considered, while these substances can, of course, not provide the contribution to osmolality that has been described for mineral salts.

As an alternative to the neutral mineral salts, physiologically acceptable organic adjuvants can be used as isotonising agents. Water-soluble substances with a relatively low molecular weight, for example with a molecular weight of less than 300, or better still less than 200, and with a correspondingly high osmotic activity are particularly useful. Examples for such adjuvants are sugars and sugar alcohols, in particular, mannitol and sorbitol.

The amount of the selected isotonising agent used is to be determined so that an osmolality of about 150 to 350 mOsmol/l results when the content of tobramycin and the acidic adjuvant and that of other optional adjuvants contained in the preparation is taken into account. Furthermore, an osmolality in the range of about 200 to 300 mOsmol/l is preferred. In a further embodiment, the preparation has an osmolality of about 230 to 280 mOsmol/l.

The acidic adjuvant in the preparation serves several purposes simultaneously. Firstly, the pH is adjusted to a range which is physiologically well tolerated (an aqueous solution of tobramycin base reacts basic, which is unfavourable for inhalation). For reasons of compatibility, the preparation should, however, be adjusted to a pH of about 5.0 to 7.0, preferably to a pH of 5.5 to 6.5.

Secondly, a pH in the aforementioned range of 5.0 to 7.0 or of 5.5 to 6.5 is particularly advantageous with respect to the physicochemical and chemical properties of the preparation, in particular with respect to the chemical stability of the active agent contained therein. If a compromise between a particularly high stability and an acceptable compatibility is desired, a pH in the acidic region down to about pH 4.0 might also be selected. The use of the acidic adjuvant entails that the active agent tobramycin is present in the preparation, at least in part, as a salt.

Particularly useful adjuvants for lowering the pH are strong mineral acids, in particular, sulfuric acid and hydrochloric acid. Furthermore, inorganic or organic acids of intermediate strength as well as acidic salts may be used, for example, phosphoric acid, citric acid, tartaric acid, succinic acid, fumaric acid, lysine, methionine, acidic hydrogen phosphates with sodium or potassium, lactic acid etc. However, sulfuric acid and hydrochloric acid are most preferred.

The preparation can optionally contain a surface active substance as an adjuvant. Surfactants are used in liquid pharmaceutical preparations in order to stabilise dispersed solid or liquid particles or in order to solubilise a—usually rather poorly soluble—active agent colloidally, for example, in the form of micelles or as a so-called microemulsion. Surfactants may be useful in order to achieve a particular surface tension, which is of great importance for optimal and reproducible nebulisation.

In a preferred embodiment, the preparation according to the invention has a surface tension of about 70 to 76 mN/m under standard conditions, i.e., at room temperature and under normal pressure. In a further embodiment, the preparation has a surface tension of at about 72 mN/m.

These surface tensions facilitate efficient nebulisation with a high proportion of respirable droplets with a diameter of at most 5 μm by means of common nebulisers. These may also be achieved without addition of a surfactant.

However, if the preparation is to be adjusted for use in a particular type of nebuliser, the surface tension may be reduced to values below about 70 mN/m, optionally even to values below about 55 mN/m at room temperature. Even when surfactants are added, the surface tension should not be lower than about 30-35 mN/m. The surface tension lowered by surfactants can be helpful in improving the spreadability of the aerosols in the lung, which may, in turn, have a positive influence on the effectiveness of the application.

As the inventors have surprisingly found, the addition of surface active substances can have a further advantageous effect: The sensory quality, i.e., in particular, the taste of the preparation upon inhalation, may be improved by suitable surfactants. However, the surfactants must be pharmaceutically acceptable and suitable for pulmonary application. Examples for such surfactants are Tweens® (in particular Tween® 80), tyloxapol, vitamin E TPGS and phospholipids, such as hydrogenated lecithins.

The amount used depends on the intended effect: If an improved spreadability in the lungs is primarily intended, the use of Tween® 80 and phospholipids in relatively high concentrations of about 0.01 to 0.1% (w/v) will be particularly useful. If, in addition thereto, the taste of the active agent is to be masked, the use of slightly higher concentrations, for example of about 0.2 to 2% (w/v) is to be preferred. A combination of tyloxapol and a phospholipid such as dimyristoyl phosphatidyl choline (DMPC) is particularly preferred and the concentration of tyloxapol should be about 0.5 to 1.5% (w/v), most preferably about 1.0% (w/v) and that of DMPC or a comparable phospholipid should be about 0.2 to 1.0% (w/v), particularly preferred about 0.5% (w/v). Surprisingly, such a combination of surfactants does not result in an increase, but rather to a decrease in local irritations in the respiratory tract or in bronchoconstrictions.

The combination of tyloxapol and a phospholipid, in particular DMPC, in connection with the active agent of tobramycin is particularly useful also from a galenic point of view. Tyloxapol alone has only limited compatibility with the active agent, i.e., precipitations result within the desirable concentration ranges of tyloxapol and tobramycin. This incompatibility, however, can be reduced or eliminated completely by the combination with DMPC.

Depending on the type and configuration of the nebuliser, the density of the preparation, too, can have an influence on the efficiency of nebulisation. It should be between about 1.0 and 1.2 g/ml, preferably between about 1.05 and 1.1 g/ml, for example 1.07 g/ml.

The manufacture of a preparation according to the invention can be carried out, for example, by dissolving the acidic adjuvant, the active agent and the isotonising agent, one after the other, under aseptic conditions in a measured amount of water for injection. Depending on whether and which surfactants are to be used, a step of homogenisation may optionally have to be carried out after their addition. In a preferred embodiment, the method of manufacture comprises the cooling of the solution during or in close temporal relationship to the dissolution of the active agent in the aqueous phase. By this measure, the active agent can be further stabilised and protected against degradation. For reasons of stability, it may also be advantageous to work under a protective atmosphere.

The filling is preferably carried out under aseptic conditions in single or multiple dose containers. Suitable primary packagings are, for example, polypropylene or polyethylene vials (PP/PE vials) and cycloolefin copolymer blisters (COC blisters). Seals plastic containers such as PP or PE vials can be formed, filled and sealed, for example, preferably by the blow fill seal method in an integrated process. The container thus produced are particularly useful for liquid products with a volume from about 0.2 ml. In a particularly patient-friendly embodiments, they can be formed with a closure which can be removed by turning or bending. The opening thus formed, through which the liquid content can be removed, can be designed so that it fits to a Luer connection or a Luer lock connection. The opening can be round and have a diameter which essentially corresponds to the external diameter of a male Luer connection. In this way, a conventional syringe with a Luer connection may be connected tightly to the container, for example, in order to take up the contents of the container and transfer them to a nebuliser or in order to mix the contents of the container with the contents of the syringe and subsequently transfer them to the nebuliser. As a further alternative, the plastic container may be designed such that, after removal of the closure element, it can be connected essentially tightly to a connection element for the input of liquid of a correspondingly adapted nebuliser whereby the reservoir of the inhaler can be filled directly with the preparation.

Plastic containers of this kind are also advantageous because they can easily be provided with embossings. This makes paper labels redundant, which is desirable in order to avoid the migration of components of the adhesive, the paper or the printing ink through the container's wall into the preparation. Furthermore, by such an embossing, important information may be made available to visually impaired patients. The embossing can contain various information, for example, a batch designation, a use-by date, a product designation, advice for application or one or more dosage markings. Especially in the case of pediatric patients where flexible dosing depending on the age and height are frequently desirable, a multiplicity of volume markings may serve to facilitate the removal of a desired dose without further implements whereby the risk of dosing errors can be reduced.

In a further embodiment of the invention, there are provided pharmaceutical kits which contain two liquid components or, alternatively, a solid and a liquid component in separate primary packagings within a common secondary packaging, wherein the components are adjusted to each other so that by combining and mixing them a tobramycin preparation according to the invention as described above, which is ready for use, can be prepared. The liquid component or one of the liquid components contains solvent and optionally further adjuvants contained therein, while the solid component (or the other liquid component) contains the active agent (tobramycin) in concentrated and stabilised form. Such kits can have the advantage of particularly great pharmaceutical stability and storability, but may still be very easy to handle and thus contribute to patient-friendliness. Alternatively, the kits may be designed such that preparations ready for use may be prepared therefrom by medically or pharmaceutically trained personnel (for example in a hospital pharmacy).

According to a further variant of the invention, there are provided multiple dose containers which contain a preparation as described above and which are designed such that the aseptic withdrawal of a single dose is possible. Thus, the multiple dose container can be a glass or plastic container like a vial or an infusion bottle with an elastomer closure which can be pierced with a syringe or it can be a complex container with dosing and withdrawal devices.

One of the particular advantages of multiple dose containers in connection with tobramycin preparations for inhalation lies in the flexibility which makes it possible to easily adjust the dose to individual needs without having to discard considerable amounts of the preparation as would be the case with single dose containers after these have been opened. In hospitals and care institutions patients can thus be cared for both particularly efficiently and potentially cost-efficiently by individual dose adjustment. In the same way, special therapeutical requirements of individual patients can easily be taken into account.

In principle, any nebuliser used in therapy can be employed to aerosolise the preparation. The well tried jet nebulisers are, in principle, as useful as more recent ultrasonic or piezoelectric nebulisers, but they are disadvantageous with respect to the inhalation time. The advantage of jet nebulisers lies in the fact that they are already very commonly available and can be obtained cost-efficiently. Many patients are already used to handling common jet nebulisers. Some modern jet nebulisers (for example, PARI LC PLUS® and PARI LC STAR®) use mechanisms by which the nebulisation is adjusted to the breathing pattern of the patient so that as large as possible a proportion of the aerosol produced is available for inhalation.

Aerosolisation of the preparation by means of a modern piezoelectric nebuliser, in particular by nebulisers of the eFlow™ type of PARI, is particularly preferred. The special advantage for patients when using this device (or a similar device) lies in the considerably shorter inhalation time compared to alternative methods. This device does not only aerosolise a greater amount of liquid per time unit, but it also produces an aerosol of particularly high quality with a high proportion of small respirable aerosol droplets.

Therapeutic success is critically dependent on the reliable and adequate availability of the active agent in the lungs. It is patient compliant to achieve this within an acceptable period of time. Patients prefer short inhalation times and inhalation times of more than about 6-8 minutes can already have a negative impact on patient compliance. Inhalation time of more than about 10 minutes are particularly undesirable. Inhalation times of less than about 5-6 minutes, on the contrary, are particularly desirable from the patients' point of view.

In conventional therapy with tobramycin, the medicament TOBI, which contains 300 mg of tobramycin in 5 ml of aqueous solution, is nebulised with the jet nebuliser recommended in the instructions for use, the PARI LC PLUS® device, which, in practice, requires a fairly long time of about 15-20 minutes. The fraction of respirable aerosol droplets with a diameter of less than 5 μm in this therapy is about 60% of the aerosol produced (measured by laser diffraction with a MasterSizer X of Malvem). Taking into account all losses of active agent occurring in the nebuliser, by exhalation by the patient and by deposition of the aerosol in the upper parts of the respiratory tract, it can be assumed that only about 60-80 mg of tobramycin reach the lungs of the patient (respirable dose, RD).

Preparations of the present invention in combination with a piezoelectric nebuliser, on the contrary, achieve markedly higher output rates. This applies, in particular, to nebulisation with a vibrating membrane nebuliser of the eFlow™ type, which, at the same time, produces higher fractions of respirable aerosol droplets, namely about 75%. Moreover, the design-related losses within the device are smaller than in the case of a jet nebuliser. Therefore, a lower amount of active agent is sufficient in order to make the same dose of active agent available in the lungs. Thus, it can be assumed, for example on the basis of in vitro data, that a preparation according to the present invention with only 200 mg of tobramycin in 2 ml of inhalation solution, upon nebulisation with the eFlow™ device, results in an availability of about 70-80 mg of active agent in the lungs (respirable dose, RD), i.e., should be bioequivalent to the conventional therapy with 5 ml TOBI 300 mg. The special advantage to patients lies in the short time required for inhalation of the 2 ml of the preparation according to the present invention with the eFlow™ device: In an experimental in vitro set-up, this takes place within about 3-4 minutes, while in practice, about 4-5 minutes are required and, in any case, less than 6 minutes, which constitutes a marked difference compared to the conventional therapy.

Therefore, it is preferred according to the present invention to formulate the preparation with respect to its pharmaceutical and, in particular, physicochemical parameters for optimal nebulisation with a piezoelectric nebuliser or vibrating membrane nebuliser, such as the eFlow™ device, so as to provide a particularly great benefit to patients in terms of the significantly reduced inhalation time.

In a further embodiment, the preparation is adjusted for application as an aerosol for the treatment of the upper respiratory tract. In this case, too, it is possible to locally treat infections with tobramycin-sensitive pathogens. In particular, the mucosa of the nasal and oral cavity as well as those of the paranasal, maxillary and frontal sinuses are, in principle, amenable to aerosol therapy. The oral and nasal mucosa are most easily reached by the aerosol. In this case, mechanical atomisers such as those frequently used for nasal and oral sprays can be employed. Especially adapted jet, ultrasonic or piezoelectric nebulisers can, however, be used for significantly improved wetting of the oral or nasal mucosa with the aerosolised preparation.

Efficient application of an aerosol to the less well ventilated cavities of the upper respiratory tract is more difficult. However, the frontal and paranasal sinuses are frequently the site of an infection. Usually, it will be attempted to treat such infections with expectorants and decongestant agents, which is not always successful. Serious cases are additionally treated by systemic antibiotic therapy, which, however, is not well tolerated by all patients.

The simple nasal inhalation of an aerosolised active agent preparation does conduct this into the vicinity of the sinuses; however, the predominant proportion of the aerosol passes the openings of the sinuses (ostia) without any significant proportion thereof entering the sinuses.

However, especially adapted jet nebulisers have recently become available by means of which the sinuses can be reached much better than previously. These nebulisers have a nose piece in order to direct the aerosol current into the nose. If only one nostril is used for inhalation of the aerosol, the other nostril must be closed by a suitable device. Furthermore, these nebulisers are characterised in that they produce an aerosol with a pulsating pressure. The pulsating pressure results in increased ventilation of the sinuses so that a simultaneously inhaled aerosol can spread more efficiently into these cavities. Examples for such nebulisation devices are described in DE 102 39 321 B3. In a preferred embodiment, the preparation according to the present invention is used for the manufacture of a medicament for application by means of one of the devices described therein for the treatment of infections of the upper respiratory tract, in particular, with a device of the PARI Sinus type.

### EXAMPLES

The following examples serve to illustrate the invention by way of a number of selected embodiments.

### Example 1

### Preparation of a Tobramycin Inhalation Solution with a Content of 100 mg/ml

11.08 g of tobramycin, 5.41 g of sulfuric acid (96%), 0.2 g of sodium chloride and 90.95 g of water for injection are used as starting materials. All steps are carried out under aseptic conditions and under nitrogen gas. The water is provided first, to which the sulfuric acid is added. Then sodium chloride and the active agent are added one after the other. The mixture is stirred until complete dissolution of all solid components as determined by visual control. This yields about 100 ml of a solution which has a pH of about 6.0, an osmolality of about 0.22 Osmol/l, a dynamic viscosity of about 1.9 mPa·s and a surface tension of about 71 mN/m. This solution is filtered to sterility and filled into an infusion bottle with a volume of 100 ml. This bottle is tightly closed with a pierceable elastomer stopper and secured with an aluminium cap.

### Example 2

### Nebulisation of a Tobramycin Inhalation Solution with a Content of 100 mg/ml with a Piezoelectric Nebuliser

2 ml of the solution prepared according to Example 1 are withdrawn aseptically with a sterile canula and a syringe and added to the reservoir of a piezoelectric nebuliser of the eFlow™ type (PARI). The device was operated according to the instructions for use in order to produce an aerosol. The aerosol was examined for respirability by means of laser diffraction (Malvern MasterSizer X) and in an Andersen cascade impactor. Nebulisation required 3.2 minutes. The fraction of particles up to 5 μm determined by laser diffraction was 75%, the fraction up to 5 μm determined by cascade impactor was 77%.

### Example 3

### Preparation of a Surfactant-Containing Tobramycin Inhalation Solution

10.88 g of tobramycin, 5.41 g of sulfuric acid (96%), 0.2 g sodium chloride, 0.1 g Tween® 80 and 90.95 g of water for injection are used as starting materials. All steps take place under aseptic conditions and nitrogen gas. The water is provided first to which the sulfuric acid is added. Subsequently, tobramycin is added and dissolved at room temperature. Sodium chloride and Tween® are added to this solution. The mixture is stirred until a clear solution is formed. This yields about 100 ml of a solution which has a pH of about 6.2, an osmolality of about 0.22 Osmol/l, a dynamic viscosity of about 1.9 mPa·s and a surface tension of about 43 mN/m. The solution is filtered to sterility and filled aseptically into single dose containers of polypropylene at 2 ml each.

### Example 4

### Preparation of a tobramycin inhalation solution with 2 surfactants

10.88 g of tobramycin, 5.41 g of sulfuric acid (96%), 0.2 g of sodium chloride, 0.45 g of DMPC, 0.91 g of tyloxapol and 89.59 g of water for injection are used as starting materials. DMPC and tyloxapol are dispersed in the water first. This mixture is then homogenised under high pressure of 1500 bar until an opalescent solution is formed. Subsequently, the sulfuric acid and the active agent are added which, initially, results in the formation of a precipitate which, however, is no longer observed after stirring for 24 h at room temperature when the mixture is no more than opalescent. Finally, the sodium chloride is added, the solution is filtered to sterility and filled into single dose containers. The solution has a pH of about 6.2, a surface tension of about 36.5 mN/m, a dynamic viscosity of about 2.07 mPa·s and an osmolality of about 0.23 Osmol/l.

### Example 5

### Preparation of a Tobramycin Inhalation Solution with Addition of Cacl₂

10.88 g of tobramycin, 5.41 g of sulfuric acid (96%), 0.2 g of sodium chloride, 0.07 g of calcium chloride and 90.95 g of water for injection are used as starting materials. The water is provided first into which the sulfuric acid is added. Subsequently, the active agent and then the sodium chloride together with the calcium chloride are added after each other. The mixture is stirred until complete dissolution of all solid components which is controlled visually. The addition of the salts may result in a transient precipitation which is no longer observed after stirring for 12 hours. The solution is filtered to sterility and filled into single dose containers. The preparation has a pH of about 6.0, a surface tension of about 70.2 mN/m, a viscosity of about 1.87 mPa·s and an osmolality of about 0.24 Osmol/kg.

### Example 6

### Preparation of a tobramycin inhalation solution with addition of MgSO₄

10.88 g of tobramycin, 5.41 g of sulfuric acid (96%), 0.2 g of sodium chloride, 0.12 g of magnesium sulphate heptahydrate and 90.95 g of water for injection are used as starting materials. The preparation of the solution is carried out as in Example 5. The solution is filtered to sterility and filled into single dose containers. The preparation has a pH of about 6.1, a surface tension of about 69.8 mN/m, a viscosity of about 1.86 mPa·s and an osmolality of about 0.24 Osmol/kg.

### Example 7

### Nebulisation of a Tobramycin Inhalation Solution with a Vibrating Membrane Nebuliser and Characterisation of the Aerosol in a Cascade Impactor and Breath Simulator

1.4 ml of a tobramycin solution according to the invention prepared as in Example 1 are withdrawn and nebulised with a piezoelectric nebuliser (vibrating membrane nebuliser) of the eFlow™ type (PARI GmbH) and the aerosol is characterised in an Anderson cascade impactor (ACI) and a breath simulator of the PARI COMPAS™ type (15 breaths/min, 500 ml tidal volumes, ratio inhalation:exhalation 1:1) and compared with the nebulisation of a commercially available tobramycin solution (TOBI™ 300, 5 ml) in a jet nebuliser of the PARI LC PLUS® type. Moreover, the geometric droplet size distribution of the aerosols was determined by photon correlation spectroscopy (PCS) using a Malvern MasterSizer X. The results are summarised in Table 1.

TABLE 1

| | According to | | TOBI 300 mg | |
|---|---|---|---|---|
| | Example 6 | | (5.0 ml) | |
| | (1.4 ml) | | PARI LC | |
| | PARI eFlow ™ | | PLUS ® | |
| Inhalation solution | | SD | | SD |
| Nebuliser | average | (n = 3) | average | (n = 3) |
| MMD (PCS) [μm] at 20 l/min | 3.89 | 0.10 | 3.90 | 0.05 |
| GSD [μm] | 1.50 | 0.01 | 2.02 | 0.02 |
| FPF [% < 5 μm] | 73.8 | 2.1 | 64.4 | 0.6 |
| TOR [mg/min] | 521.2 | 45.7 | 465.3 | 13.3 |
| MMAD (ACI) [μm] at 28.3 l/min | 3.89 | 0.14 | 3.39 | 0.12 |
| GSD [μm] | 1.48 | 0.04 | 2.15 | 0.01 |
| FPF [% < 5 μm] | 71.5 | 0.5 | 71.1 | 0.1 |
| DD [mg] | 95.3 | 1.78 | 112.9 | 4.10 |
| Loss of active agent in the | 12.0 | 0.90 | 133.2 | 7.87 |
| nebuliser [mg] | | | | |
| Loss of active agent upon | 26.4 | 2.91 | 52.3 | 4.52 |
| nebulisation [mg] | | | | |
| Duration of nebulisation | 3.0 | 0.22 | 15.3 | 1.50 |
| [min] | | | | |
| DD [% of the dose] | 68.9 | 1.26 | 37.2 | 1.36 |
| Loss of active agent in | 8.7 | 0.65 | 43.9 | 2.59 |
| the nebuliser [% of the dose] | | | | |
| Loss of active agent upon | 19.0 | 2.10 | 17.3 | 1.49 |
| nebulisation [% of the dose] | | | | |
| Balance [% of the dose] | 96.6 | 0.68 | 98.4 | 0.67 |
| RD [% of DD < 5 μm] | 70.3 | 2.52 | 72.7 | 3.10 |
| RD [% of dose < 5 μm] | 50.8 | 1.82 | 24.0 | 1.03 |
| DDR [mg/min] | 31.4 | 2.27 | 7.4 | 0.78 |
| DDR [% of dose/min] | 22.7 | 1.65 | 2.5 | 0.26 |
| RDDR [mg < 5 μm/min] | 23.1 | 1.13 | 4.8 | 0.50 |

Explanations:
MMD: mass median diameter
GSD: geometric standard deviation
FPF: fine particle fraction <5 μm
TOR: total output rate
MMAD: mass median aerodynamic diameter
DD: delivered dose
RD: respirable dose
DDR: drug delivery rate
RDDR: respirable drug delivery rate

1-24. (canceled)

25. A sterile, liquid preparation in the form of an aqueous solution for the application as a solution for injection or as an aerosol containing about 80 mg/ml to 120 mg/ml of tobramycin and an acidic adjuvant, wherein the preparation comprises not more than 2 mg/ml of sodium chloride.

26. The preparation according to claim 25 wherein the preparation is substantially free of sodium chloride.

27. The preparation according to claim 26 wherein the preparation contains at least one substantially neutral isotonising agent.

28. The preparation according to claim 27 wherein the isotonising agent is a magnesium salt, a calcium salt, a sugar or a sugar alcohol.

29. The preparation according to claim 25 wherein the preparation has a pH of about 5.5 to about 6.5.

30. The preparation according to claim 25 wherein the acidic adjuvant is sulfuric acid or hydrochloric acid.

31. The preparation according to claim 25 wherein the preparation contains at least one surface active adjuvant.

32. The preparation according to claim 31 wherein the surface active adjuvant is a phospholipid.

33. The preparation according to claim 32 wherein the preparation contains tyloxapol as a further surface active adjuvant.

34. The preparation according to claim 25 wherein the preparation has a dynamic viscosity at room temperature of about 1.6 to 2.0 mPas and an osmolality of about 200 to 300 mOsmol/l.

35. The preparation according to claim 25 wherein the preparation has an osmolality of about 230 to 280 mOsmol/l.

36. The preparation according to claim 25 wherein the preparation exists as a measured single dose within a primary packaging.

37. The preparation according to claim 36 wherein the primary packaging is formed by a plastic container which comprises a removal closure element.

38. The preparation according to claim 37 wherein the removal of the closure element forms a round opening in the plastic container, the diameter of which corresponds to about the internal diameter of a female Luer lock adapter.

39. The preparation according to claim 37 wherein the plastic container, after removal of the closure element, can be fitted essentially tightly to the connector of a nebuliser which is provided for the input of liquid.

40. The preparation according to claim 37 wherein the plastic container is provided with at least one embossing, which represents a product designation, a lot code, a use-by date and/or a volume or dose marking.

41. A kit for the manufacture of a preparation according to claim 25, the kit comprising (a) a liquid or solid component containing an active agent and (b) a liquid component which is free of active agent.

42. The preparation according to claim 25 wherein the preparation is adapted for intravenous, intraarterial, subcutaneous or intramuscular injection.

43. The preparation according to claim 25 wherein the preparation is adapted for aerosol application.

44. The preparation according to claim 25 wherein the preparation is adapted for application by a jet, ultrasonic or piezoelectric nebuliser.

45. The preparation of claim 44 wherein the preparation is adapted for application by a piezoelectric nebuliser.

46. The preparation of claim 45 wherein the piezoelectric nebuliser is a device of the eFlow™ type of PARI.

47. The preparation of claim 25 wherein the preparation is adapted for nasal application by a mechanical atomiser or a jet, ultrasonic or piezoelectric nebuliser.

48. The preparation of claim 47 wherein the preparation is adapted for administration to the mucosa of the paranasal and/or frontal sinuses.

49. The preparation according to claim 47 wherein the preparation is adapted for administration by a jet nebuliser which comprises a nose piece for supplying an aerosol to one or both nostrils of a patient and the aerosol output of which has a pulsating pressure.

50. A method for treating a subject comprising administering a preparation of claim 25 to the subject by aerosol application.

51. A method for treating a subject comprising administering a preparation of claim 25 to the subject by intravenous, intraarterial, subcutaneous or intramuscular injection.

52. A method for treating a subject comprising nasally or pulmonarily administering a preparation of claim 25 to the subject.

53. The method of claim 52 wherein the preparation is administered nasally.

54. The method of claim 52 wherein the preparation is administered pulmonarily.

55. A method for treating a subject comprising administering a preparation of claim 25 to the subject by a jet, ultrasonic or piezoelectric nebuliser.
